(19) United States
(12) Patent Application Publication
(10) Pub. No.: US 2023/0400428 A1
(43) Pub. Date: Dec. 14, 2023

(54) SOIL LANDSLIDE MATRIX SUCTION TESTING METHOD AND SYSTEM BASED ON SOIL BODY CONDUCTIVITY

(71) Applicants: Fuzhou University, Fuzhou (CN); Fuzhou Planning & Design Research Institute Group Co., Ltd, Fuzhou (CN)

(72) Inventors: Wenbin JIAN, Fuzhou (CN); Yunzhao LIN, Fuzhou (CN); Chang XIA, Fuzhou (CN); Ruimin CHEN, Fuzhou (CN); Xin ZHONG, Fuzhou (CN); Hongqiang DOU, Fuzhou (CN); Xiufeng FAN, Fuzhou (CN)

(21) Appl. No.: 18/332,691

(22) Filed: Jun. 9, 2023

(30) Foreign Application Priority Data
Jun. 13, 2022 (CN) 202210673547.2

(51) Int. Cl. G01N 27/04; G01N 33/24

(52) U.S. Cl. CPC G01N 27/041; G01N 27/048; G01N 33/246

(57) ABSTRACT

A soil landslide matrix suction testing method and system based on soil body conductivity. The method comprises: establishing a calculation formula of an unsaturated residual soil matrix suction measurement method based on soil body conductivity; acquiring actual measurement data of an unsaturated soil conductivity-moisture content curve measured by an indoor soil slope rainfall experiment; acquiring actual measurement data of an unsaturated soil moisture content-matric suction curve measured by an indoor soil slope rainfall experiment; substituting the unsaturated soil conductivity data and the unsaturated soil matrix suction data measured into the calculation formula for fitting to obtain a saturation index and fitting parameters; and substituting the saturation index and the fitting parameters obtained into the calculation formula to obtain a conductivity-matric suction model of the soil body, and substituting a conductivity of an unsaturated soil body into the conductivity-matric suction model to obtain a matric suction of the soil body.

### CROSS REFERENCE TO RELATED APPLICATIONS

This application claims foreign priority of Chinese Patent Application No. 202210673547.2, filed on Jun. 13, 2022 in the China National Intellectual Property Administration, the disclosures of all of which are hereby incorporated by reference.

### TECHNICAL FIELD

The present invention belongs to the technical field of geotechnical and geological engineering, and particularly relates to a soil landslide matrix suction testing method and system based on soil body conductivity.

### BACKGROUND OF THE PRESENT INVENTION

Rainfall is the most important and most common environmental factor influencing side slope stability to cause slope instability. One non-negligible factor in the influence of moisture infiltration on landslide stability is the matric suction change of a soil body. With the moisture infiltration, matrix suction of a side slope unsaturated area is reduced, so that a shear strength of a soft interlayer of the soil body or a rock mass is greatly reduced, and thus, the side slope stability is reduced. The matrix suction is an important variable to characterize properties of the unsaturated soil. As a component of soil body cohesion, the matrix suction can improve the shear strength of the soil body and contribute to the side slope stability. A soil-water characteristic curve shows that the matrix suction has strong water sensitivity. With the increase of the moisture content, the matrix suction decreases rapidly. In practical engineering, most side slopes are in unsaturated state or partially saturated state. Under the action of rainfall, with the rainwater infiltration, the matric suction of the soil body decreases rapidly and the cohesion decreases, thus reducing the side slope stability. Therefore, in landslide early warning, the matrix suction is an important monitoring index, but large-scale monitoring means of the matrix suction are still not diverse and convenient enough.

The conductivity of soil is an important physical property of soil, and is related to a moisture content of soil and a particle size of soil. Because it is convenient, fast and cheap to test the conductivity of soil, the conductivity of soil has also been applied in side slope research. Experimental studies show that the overall distributions of the side slope conductivity and the matrix suction are similar, and there is a strong correlation between the side slope conductivity and the matrix suction. As an effective index for reflecting an occurrence state of the medium water, and benefiting from more diverse and convenient detection means of the conductivity, for a natural side slope with complex and changeable water distribution characteristics and migration rules, it is an effective method to feedback the distribution characteristics of related parameters such as the moisture content and the matrix suction by using the conductivity of side slope medium for natural slopes with complex and changeable water distribution characteristics and migration laws.

### SUMMARY OF PRESENT INVENTION

The present invention aims at providing a soil landslide matrix suction testing method and system based on soil body conductivity, which are beneficial to conveniently and effectively measuring matric suction of a soil body.

In order to achieve the above objects of the present invention, the following technical solutions are employed in the present invention. A soil landslide matrix suction testing method based on soil body conductivity comprises the following steps of:
1) establishing an unsaturated residual soil matrix suction measurement method based on soil body conductivity:

$$ψ = {\frac{1}{α} [ {( \frac{{σ}_{s}}{σ} )}^{\frac{β}{p ( β - 1 )}} - 1 ]}^{\frac{1}{β}} \qquad ( 1 )$$

wherein in the formula, σ is soil body conductivity, σₛ is saturated soil body conductivity, ψ is matrix suction, α and β are fitting parameters, and p is a saturation index;
2) acquiring actual measurement data of an unsaturated soil conductivity-moisture content curve measured by an indoor soil slope rainfall experiment;
3) acquiring actual measurement data of an unsaturated soil moisture content-matric suction curve measured by an indoor soil slope rainfall experiment;
(4) substituting the unsaturated soil conductivity data measured in the step 2) and the unsaturated soil matrix suction data measured in the step 3) into the formula (1) for fitting to obtain the saturation index p and the fitting parameters α and β; and
5) substituting the saturation index p and the fitting parameters α and β obtained into the formula (1) to obtain a conductivity-matric suction model of the soil body, and substituting a conductivity of an unsaturated soil body into the conductivity-matric suction model to obtain a matric suction of the soil body.

Further, the step 1) specifically comprises the following steps of:
1a) expanding an Archie model to unsaturated soil, and establishing a soil body saturation-resistivity relationship:

ρ=αρ_{w}n^{-m}Sᵣ^{-p} (2)
wherein in the formula, ρ is soil body resistivity, ρ_{w} is pore water resistivity, α is a soil property parameter, m is a soil property parameter, n is soil body porosity, Sᵣ is saturability, and p is a saturation index;
1b) the soil conductivity being a reciprocal of the resistivity:

$$σ = \frac{1}{ρ} \qquad ( 3 )$$

1c) combining the formula (2) and the formula (3) to obtain the soil body saturability-conductivity relationship:

$$σ = \frac{{σ}_{w}}{a} \cdot {n}^{m} {S}_{r}^{p} \qquad ( 4 )$$

wherein in the formula, σ is the soil body conductivity, and as is the saturated soil body conductivity;
1d) a conversion relationship between a volume moisture content of soil and the saturation of soil being as follows:

$$θ = \frac{{V}_{W}}{V} = w \frac{{γ}_{d}}{{γ}_{W}} = n {S}_{r} \qquad ( 5 )$$

wherein in the formula, θ is a volume moisture content, V_{w} is a pore water volume, Vis a total volume of soil sample, w is a moisture content, γ_{d} is a dry weight of soil, and γ_{w} is a pore water weight;
1e) combining the formula (4) and the formula (5), substituting a conversion relationship formula between the volume moisture content of soil and the soil body and density into a conversion relationship between the saturation of soil and the moisture content to obtain a soil body conductivity-moisture content model:

$$σ = \frac{{σ}_{w}}{a} {n}^{m - p} {θ}^{p} \qquad ( 6 )$$

1f) in the unsaturated soil, a relationship curve between the soil suction and the volume moisture content being called a soil-water characteristic curve, which is expressed by a Van Genuchten model as follows:

$$θ = {θ}_{r} + \frac{{θ}_{s} - {θ}_{r}}{{( 1 + {( α ψ )}^{β} )}^{\frac{β - 1}{β}}} \qquad ( 7 )$$

wherein in the formula, θₛ θₛ and θᵣ are a saturated volume moisture content and a residual volume moisture content respectively;
regardless of an influence of the residual moisture content, the VG model being modified as follows:

$$θ = \frac{{θ}_{s}}{{( 1 + {( α ψ )}^{β} )}^{\frac{β - 1}{β}}} \qquad ( 8 )$$

1g) replacing a volume moisture content parameter in Van Genuchten model parameters with the conductivity in the conductivity-volume moisture content formula, and combining the formula (6) and the formula (8) to obtain a mathematical model between the matrix suction and the conductivity as follows:

$$ψ = {\frac{1}{α} [ {( \frac{{σ}_{s}}{σ} )}^{\frac{β}{p ( β - 1 )}} - 1 ]}^{\frac{1}{β}} . \qquad ( 1 )$$

Further, in the step 4), the method of fitting the saturation index p and the fitting parameters α and β specifically comprises: based on the unsaturated soil conductivity data and the unsaturated soil matrix suction data measured, drawing a scatter plot with the conductivity as an abscissa and the matrix suction as an ordinate, and then performing nonlinear fitting to obtain the saturation index p and the fitting parameters α and β.

The present invention provides a soil landslide matrix suction testing system based on soil body conductivity comprising a memory, a processor and a computer program instruction stored in the memory and capable of being run by the processor, wherein when running the computer program instruction, the processor is capable of realizing the method steps above.

Compared with the prior art, the present invention has the following beneficial effects. According to the present invention, the matric suction of the soil body is measured by using soil body conductivity parameters, so that a new choice is added for measuring the matric suction of the soil body, and benefiting from more diverse and convenient detection means of the conductivity of the soil body conductivity, such as Electrical Resistivity Tomography (ERT), a problem that large-scale monitoring means of the matrix suction are still not diverse and convenient is solved. As a component of soil body cohesion, the matrix suction can improve the shear strength of the soil body and contribute to the side slope stability. Therefore, measuring the matrix suction through the soil body conductivity has important theoretical and practical significance for the stability analysis and monitoring and early warning of the soil slope.

### DESCRIPTION OF THE DRAWINGS

FIG. 1 is a flowchart of implementing a method of the embodiments of the present invention;

FIG. 2 is a sectional diagram showing instrument arrangement of an indoor soil slope rainfall experiment in the embodiments of the present invention;

FIG. 3 is a plan diagram showing the instrument arrangement of the indoor soil slope rainfall experiment in the embodiments of the present invention; and

FIG. 4 is a fitting diagram of a residual soil conductivity-matrix suction curve in the embodiments of the present invention.

### DETAILED DESCRIPTION OF PREFERRED EMBODIMENTS

The present invention will be further described in detail below with reference to the drawings and embodiments.

It should be noted that the following detailed descriptions are all exemplary and are intended to provide a further understanding of this application. Unless otherwise specified, all technical and scientific terms used herein have the same meaning as commonly understood by a person of ordinary skill in the art to which this application belongs.

It should be noted that terms used herein are only for describing specific implementations and are not intended to limit exemplary implementations according to this application. As used herein, the singular form is intended to include the plural form, unless the context clearly indicates otherwise. In addition, it should also be further understood that terms “include” and/or “comprise” used in this specification indicate that there are features, steps, operations, devices, assemblies, and/or combinations thereof.

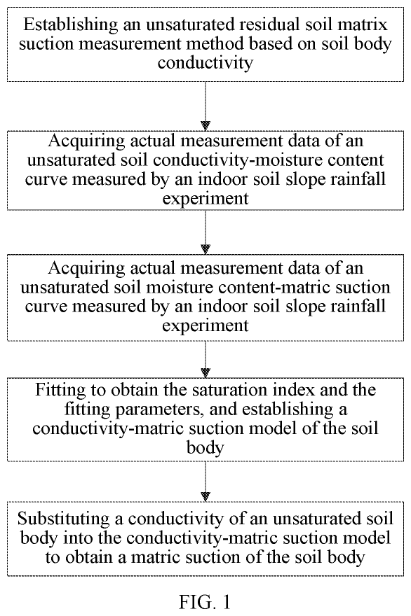

As shown in FIG. 1, the embodiment provides a soil landslide matrix suction testing method based on soil body conductivity, comprising the following steps.

1) Establishing an unsaturated residual soil matrix suction measurement method based on soil body conductivity:

$$ψ = {\frac{1}{α} [ {( \frac{{σ}_{s}}{σ} )}^{\frac{β}{p ( β - 1 )}} - 1 ]}^{\frac{1}{β}} \qquad ( 1 )$$

wherein in the formula, σ is soil body conductivity, σₛ is saturated soil body conductivity, ψ is matrix suction, α and β are fitting parameters, and p is a saturation index.

In this embodiment, the step 1) specifically comprises the following steps.

1a) Expanding an Archie model to unsaturated soil, and establishing a soil body saturation-resistivity relationship:

ρ=αρ_{w}n^{-m}Sᵣ^{-p} (2)

wherein in the formula, ρ is soil body resistivity, ρ_{w} is pore water resistivity, α is a soil property parameter, m is a soil property parameter, n is soil body porosity, Sᵣ is saturability, and p is a saturation index.

1b) The soil conductivity being a reciprocal of the resistivity:

$$σ = \frac{1}{ρ} \qquad ( 3 )$$

1c) Combining the formula (2) and the formula (3) to obtain the soil body saturability-conductivity relationship:

$$σ = \frac{{σ}_{w}}{a} \cdot {n}^{m} {S}_{r}^{p} \qquad ( 4 )$$

wherein in the formula, σ is the soil body conductivity, and σₛ is the saturated soil body conductivity.

1d) A conversion relationship between a volume moisture content of soil and the saturation of soil being as follows:

$$θ = \frac{{V}_{W}}{V} = w \frac{{γ}_{d}}{{γ}_{W}} = n {S}_{r} \qquad ( 5 )$$

wherein in the formula, θ is a volume moisture content, V_{w} is a pore water volume, V is a total volume of soil sample, w is a moisture content, γ_{d} is a dry weight of soil, and γ_{w} is a pore water weight.

1e) Combining the formula (4) and the formula (5), substituting a conversion relationship formula between the volume moisture content of soil and the soil body and density into a conversion relationship between the saturation of soil and the moisture content to obtain a soil body conductivity-moisture content model:

$$σ = \frac{{σ}_{w}}{a} = {n}^{m - p} {θ}^{p} \qquad ( 6 )$$

1f) In the unsaturated soil, a relationship curve between the soil suction and the volume moisture content being called a soil-water characteristic curve (SWCC), wherein the soil-water characteristic curve is related to soil permeability function and shear strength index. Common models comprise Van Genuchten model, Fredlund-Xing model and the like. In this embodiment, the Van Genuchten model is represented as follows:

$$θ = {θ}_{r} + \frac{{θ}_{s} - {θ}_{r}}{{( 1 + {( α ψ )}^{β} )}^{\frac{β - 1}{β}}} \qquad ( 7 )$$

wherein in the formula, θₛ and θᵣ are a saturated volume moisture content and a residual volume moisture content respectively.

Because it is difficult to determine a residual moisture content of residual soil, the influence of the residual moisture content is not considered in this embodiment, and the VG model is modified as follows:

$$θ = \frac{{θ}_{s}}{{( 1 + {( α ψ )}^{β} )}^{\frac{β - 1}{β}}} \qquad ( 8 )$$

1g) replacing a volume moisture content parameter in Van Genuchten model parameters with the conductivity in the conductivity-volume moisture content formula (Keller modifier formula), and combining the formula (6) and the formula (8) to obtain a mathematical model between the matrix suction and the conductivity as follows:

$$ψ = {\frac{1}{α} [ {( \frac{{σ}_{s}}{σ} )}^{\frac{β}{p ( β - 1 )}} - 1 ]}^{\frac{1}{β}} \qquad ( 1 )$$

2) Acquiring actual measurement data of an unsaturated soil conductivity-moisture content curve measured by an indoor soil slope rainfall experiment.

3) Acquiring actual measurement data of an unsaturated soil moisture content-matric suction curve measured by an indoor soil slope rainfall experiment.

(4) Substituting the unsaturated soil conductivity data measured in the step 2) and the unsaturated soil matrix suction data measured in the step 3) into the formula (1) for fitting to obtain the saturation index p and the fitting parameters α and β.

In the embodiment, the method of fitting the saturation index p and the fitting parameters α and β specifically comprises: based on the unsaturated soil conductivity data and the unsaturated soil matrix suction data measured, drawing a scatter plot with the conductivity as an abscissa and the matrix suction as an ordinate, and then performing nonlinear fitting to obtain the saturation index p and the fitting parameters α and β.

5) Substituting the saturation index and the fitting parameter obtained into the formula (1) to obtain a conductivity-matric suction model of the soil body, and substituting a conductivity of an unsaturated soil body into the conductivity-matric suction model to obtain a matric suction of the soil body.

The embodiment also provides a soil landslide matrix suction testing system based on soil body conductivity, comprising a memory, a processor and a computer program instruction stored in the memory and capable of being run by the processor, wherein when running the computer program instruction, the processor is capable of realizing the method steps above.

The present invention will be further described hereinafter in detail with reference to the specific embodiments.

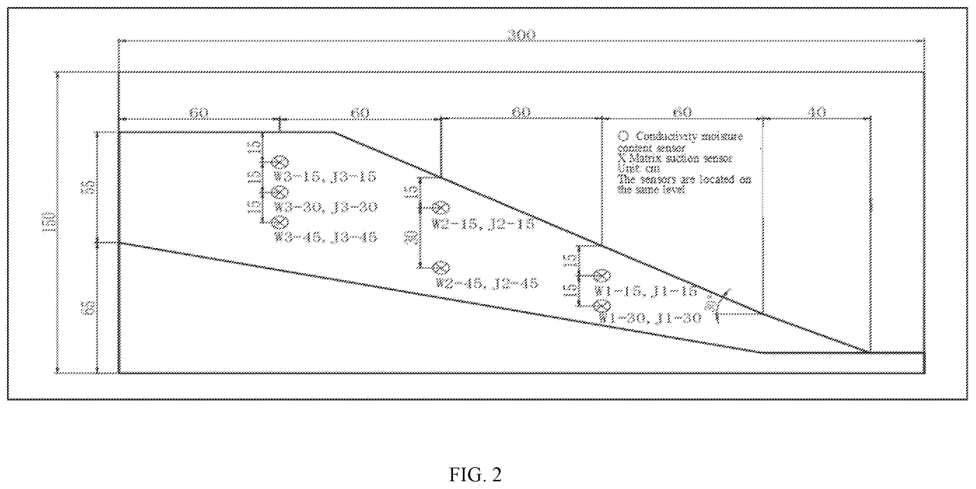

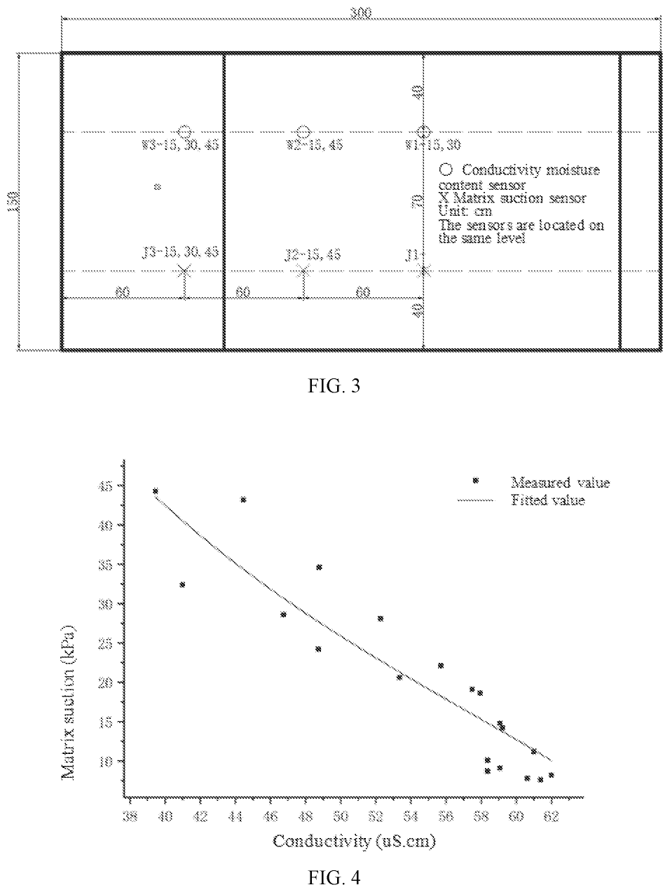

Soil samples used in the embodiment were unsaturated residual soil in Fuzhou Region, Fujian Province. Firstly, according to an indoor soil landslide rainfall experiment, a soil body conductivity moisture content sensor and a matrix suction sensor were buried in an indoor soil slope (a sectional diagram and a plan diagram of the indoor soil slope model and sensor embedding were shown in FIGS. 2 and 3) to obtain matrix suction values of the soil body with different conductivity. Moreover, the soil body conductivity was taken as the abscissa, and the matrix suction was taken as the ordinate to draw a scatter plot. Then, the data was substituted into a matrix suction measurement model based on soil body conductivity, i.e., formula (1), for nonlinear fitting to obtain saturation index p and fitting parameters α and β. A fitting result of the residual soil used in the experiment was that α=0.03, β=1.64, and p=1.31. A residual soil matrix suction prediction model based on soil body conductivity was that: ψ=33.33[(σ,σ)^{1.95}−1]^{0.61} and R²=0.84. The fitting curve was shown in FIG. 4. Some points were randomly selected from the measured data, substituted into the fitting formula for checking calculation. Measured values were compared with fitted values. An error range was reasonable and accurate, as shown in Table 1.

TABLE 1
Calculation results of conductivity-matrix suction prediction model

| | Measured value | Matrix suction values | |
|---|---|---|---|
| Conductivity | of matrix | fitted by prediction | Deviation |
| (uS · cm) | suction (kPa) | model (kPa) | ratio |
| 42.10 | 34.4 | 42.17 | 22.5% |
| 48.78 | 26.2 | 30.33 | 15.7% |
| 55.01 | 20.6 | 21.00 | 0.02% |
| 60.13 | 14.4 | 13.77 | 0.04% |
| 64.28 | 6.1 | 7.36 | 20.7% |

At present, the published model for measuring the matric suction of unsaturated soil does not take the soil body conductivity parameters as measurement indexes. According to the present invention, the matric suction of the soil body is measured by using the soil body conductivity parameters, so that a new choice is added for measuring the matric suction of the soil body, and benefiting from more diverse and convenient detection means of the conductivity of the soil body conductivity, such as Electrical Resistivity Tomography (ERT), which solves a problem that large-scale monitoring means of the matrix suction are still not diverse and convenient, and has important theoretical and practical significance for the stability analysis, monitoring and early warning of the soil slope.

It should be appreciated by those skilled in the art that the embodiments of the present application may be provided as methods, systems or computer program products. Accordingly, the embodiments of the present application can take the form of an entirely hardware embodiment, an entirely software embodiment, or a combination of software and hardware. Moreover, the embodiments of the present invention may take the form of a computer program product embodied on one or more computer usable storage media (including but not limited to disk memory, CD-ROM, optical memory, etc.) in which computer usable program codes are included.

The present application is described with reference to the flow charts and/or block diagrams of the method, device (system), and computer program products according to the embodiments of the present application. It should be appreciated that each flow and/or block in the flow charts and/or block diagrams, and combinations of the flows and/or blocks in the flow charts and/or block diagrams may be implemented by computer program instructions. These computer program instruction may be provided to a general purpose computer, a special purpose computer, an embedded processor, or a processor of other programmable data processing device to produce a machine for the instructions executed by the computer or the processor of other programmable data processing device to generate an apparatus for implementing the functions specified in one or more flows of the flow chart and/or in one or more blocks of the block diagram.

These computer program instructions may also be provided to a computer readable memory that can guide the computer or other programmable data processing device to work in a given manner, so that the instructions stored in the computer readable memory generate a product including an computer program instruction apparatus that implements the functions specified in one or more flows of the flow chart and/or in one or more blocks of the block diagram.

These computer program instructions may also be loaded to a computer, or other programmable data processing device, so that a series of operating steps are executed on the computer, or other programmable data processing apparatus to produce processing implemented by the computer, so that the instructions executed in the computer or other programmable data processing apparatus provide steps for implementing the functions specified in one or more flows of the flow chart and/or in one or more blocks of the block diagram.

The foregoing descriptions are merely preferred embodiments of the present invention, but are not intended to limit the present invention in other forms. Any person familiar with this art may make use of the technical contents disclosed above to make changes or modifications to equivalent embodiments. Any simple modifications, and equivalent changes and embellishments made to the above embodiments according to the technical essence of the present invention without departing from the contents of the present invention shall all fall within the protection scope of the present invention.

We claim:

1. A soil landslide matrix suction testing method based on soil body conductivity, comprising the following steps of:
1) establishing an unsaturated residual soil matrix suction measurement method based on soil body conductivity:
$$ψ = {\frac{1}{α} [ {( \frac{{σ}_{s}}{σ} )}^{\frac{β}{p ( β - 1 )}} - 1 ]}^{\frac{1}{β}} \qquad ( 1 )$$
wherein in the formula, σ is soil body conductivity, σₛ is saturated soil body conductivity, ψ is matrix suction, α and β are fitting parameters, and p is a saturation index;
2) acquiring actual measurement data of an unsaturated soil conductivity-moisture content curve measured by an indoor soil slope rainfall experiment;
3) acquiring actual measurement data of an unsaturated soil moisture content-matric suction curve measured by an indoor soil slope rainfall experiment;
(4) substituting the unsaturated soil conductivity data measured in the step 2) and the unsaturated soil matrix suction data measured in the step 3) into the formula (1) for fitting to obtain the saturation index p and the fitting parameters α and β; and
5) substituting the saturation index p and the fitting parameters α and β obtained into the formula (1) to obtain a conductivity-matric suction model of the soil body, and substituting a conductivity of an unsaturated soil body into the conductivity-matric suction model to obtain a matric suction of the soil body.

2. The soil landslide matrix suction testing method based on soil body conductivity according to claim 1, wherein the step 1) specifically comprises the following steps of:
1a) expanding an Archie model to unsaturated soil, and establishing a soil body saturation-resistivity relationship:
ρ=αρ_{w}n^{-m}Sᵣ^{-p} (2)
wherein in the formula, ρ is soil body resistivity, ρ_{w} is pore water resistivity, α is a soil property parameter, m is a soil property parameter, n is soil body porosity, Sᵣ is saturability, and p is a saturation index;
1b) the soil conductivity being a reciprocal of the resistivity:
$$σ = \frac{1}{ρ} \qquad ( 3 )$$
1c) combining the formula (2) and the formula (3) to obtain the soil body saturability-conductivity relationship:
$$σ = \frac{{σ}_{w}}{a} \cdot {n}^{m} {S}_{r}^{p} \qquad ( 4 )$$
wherein in the formula, σ is the soil body conductivity, and σₛ is the saturated soil body conductivity;
1d) a conversion relationship between a volume moisture content of soil and the saturation of soil being as follows:
$$θ = \frac{{V}_{W}}{V} = w \frac{{γ}_{d}}{{γ}_{W}} = n {S}_{r} \qquad ( 5 )$$
wherein in the formula, θ is a volume moisture content, V_{w} is a pore water volume, Vis a total volume of soil sample, w is a moisture content, γ_{d} is a dry weight of soil, and γ_{w} is a pore water weight;
1e) combining the formula (4) and the formula (5), substituting a conversion relationship formula between the volume moisture content of soil and the soil body and density into a conversion relationship between the saturation of soil and the moisture content to obtain a soil body conductivity-moisture content model:
$$σ = \frac{{σ}_{w}}{a} {n}^{m - p} {θ}^{p} \qquad ( 6 )$$
1f) in the unsaturated soil, a relationship curve between the soil suction and the volume moisture content being called a soil-water characteristic curve, which is expressed by a Van Genuchten model as follows:
$$θ = {θ}_{r} + \frac{{θ}_{s} - {θ}_{r}}{{( 1 + {( α ψ )}^{β} )}^{\frac{β - 1}{β}}} \qquad ( 7 )$$
wherein in the formula, θₛ and θᵣ are a saturated volume moisture content and a residual volume moisture content respectively;
regardless of an influence of the residual moisture content, the VG model being modified as follows:
$$θ = \frac{{θ}_{s}}{{( 1 + {( α ψ )}^{β} )}^{\frac{β - 1}{β}}} \qquad ( 8 )$$
1g) replacing a volume moisture content parameter in Van Genuchten model parameters with the conductivity in the conductivity-volume moisture content formula, and combining the formula (6) and the formula (8) to obtain a mathematical model between the matrix suction and the conductivity as follows:
$$ψ = {\frac{1}{α} [ {( \frac{{σ}_{s}}{σ} )}^{\frac{β}{p ( β - 1 )}} - 1 ]}^{\frac{1}{β}} . \qquad ( 1 )$$

3. The soil landslide matrix suction testing method based on soil body conductivity according to claim 1, wherein in the step 4), the method of fitting the saturation index p and the fitting parameters α and β specifically comprises: based on the unsaturated soil conductivity data and the unsaturated soil matrix suction data measured, drawing a scatter plot with the conductivity as an abscissa and the matrix suction as an ordinate, and then performing nonlinear fitting to obtain the saturation index p and the fitting parameters α and β.

4. A soil landslide matrix suction testing system based on soil body conductivity, comprising a memory, a processor and a computer program instruction stored in the memory and capable of being run by the processor, wherein when running the computer program instruction, the processor is capable of realizing the method steps according to claim 1.
